(19) United States
(12) Patent Application Publication
(10) Pub. No.: US 2004/0005369 A1
(43) Pub. Date: Jan. 8, 2004

(54) Method for processing biochemical fine powders of ginseng or the like

(76) Inventors: William Chuang, Los Altos, CA (US); King Chuang, Los Altos, CA (US)

Correspondence Address: William Chuang, King Chuang, 1230 Miramonte Ave., Los Altos, CA, 94024, US

(21) Appl. No.: 10/419,204

(22) Filed: Apr. 21, 2003

(30) Foreign Application Priority Data
May 17, 2002 (TW) 091110323

(51) Int. Cl. A61K 35/78

(52) U.S. Cl. 424/728; 241/17

(57) ABSTRACT

A method for processing biochemical fine powders of ginseng or the like to obtain effective ingredients, mainly including the steps of harvesting raw material, washing and dehydrating the same; forming the raw material into a mixture of liquid and paste by using millers, rollers, roller millers and/or kneaders; filtering the mixture of liquid and paste to form a suspension; and drying the suspension to form very fine powders which contain active cells and high quality ingredients of ginseng or the like. Furthermore, additional steps of sedimentation and separation after the filtering step; keeping the raw material fresh (e.g. vacuum forming or keeping it at low temperature) after dehydrating the same; sterilization (e.g. ultra-violet light, high temperature, gamma ray radiation, etc.) during or after the drying step; concentrating the suspension (e.g. sudden cooling or low temperature low pressure treatment) after the separation step; extraction; distillation; physical separation, and ionic exchange.

### FIELD OF THE INVENTION

The present invention relates to a method for producing fine powders of ginseng or the like, particularly to a method for processing biochemical fine powders (particles) of ginseng or the like, to obtain effective ingredients.

### BACKGROUND OF THE INVENTION

The present invention relates to the processing of ginseng or the like (e.g. Chinese ginseng, Korean ginseng, panax quinquefolia, panax notoginseng, radix notoginseng, etc.), particularly ginseng or the like such as radix notoginseng which is very hard after it is dried, so that it can become very fine powders while preserving its active cells and freshness, keeping effective ingredients of high quality. In the processing of the present invention, effective ingredients can be extracted by separating suspension.

Conventionally, people mostly use the following method to handle ginseng or the like to produce powders thereof: to dry the raw material, and then to grind the same so as to form powders. The drying process mostly takes place in open space, utilizing wind or sunshine to achieve drying. This requires a large area of land, takes a long time, and the raw material needs to be moved to the ground or place to be dried and moved to factory to handle. Moreover, the raw material is susceptible to the environmental contamination such as the dust, dirt, animal discharges, and mildew; susceptible to the human contamination such as the hair or saliva of the operators; and to the loss due to rainwater, animal eating, and mildew. Furthermore, during grinding, the raw material is susceptible to metallic contamination from the grinding machine and to the loss due to grinding.

### SUMMARY OF THE INVENTION

Briefly speaking, according to the present invention, the raw material ginseng or the like, after being harvested, is washed, dehydrated, formed into a mixture of liquid and paste by using millers, rollers, roller millers and/or kneaders, and then filtered. The mixture, after being filtered, can be dried directly, or dried after being separated, to form very fine powders containing active cells so as to ensure products which are clean and which preserve the high quality and characteristics of the ginseng or the like. Moreover, some processes which can keep the raw material fresh (e.g. vacuum forming, or low temperature treatment) can be done after the above-mentioned washing and dehydrating processes. Some processes such as ultraviolet light, high temperature treatment, gamma-ray radiation can be done after or during the above mentioned drying process, to achieve the purpose of sterilization. Furthermore, after the mixture of liquid and paste is separated, the processes such as concentrating treatment (e.g. sudden cooling or low temperature, low pressure treatment), extraction with solvent, distillation, and removing heavy metallic harmful contamination with physical separation or ionic exchange treatment.

### BRIEF DESCRIPTION OF THE DRAWINGS

FIG. 1 illustrates diagrams showing at least three embodiments according to the present inventions;

FIG. 2 shows an embodiment of an equipment for drying the mixture of liquid and paste in the present invention;

FIG. 3 shows another embodiment of the equipment for drying the mixture of liquid and paste in the present invention;

FIG. 4 shows a table illustrating the comparative results according to conventional method and the method according to the present invention;

FIG. 5(a)˜5(g) show diagrams illustrating the comparative results HPLC (Current pressure liquid chromatogram) according to conventional method and the method according to the present invention;

FIG. 6 shows an embodiment of an equipment for filtering the mixture of liquid and paste according to the present invention; and

FIG. 7 shows another embodiment of the equipment for filtering the mixture of liquid and paste utilizing centrifugal force according to the present invention.

### DETAILED DESCRIPTION OF THE INVENTION

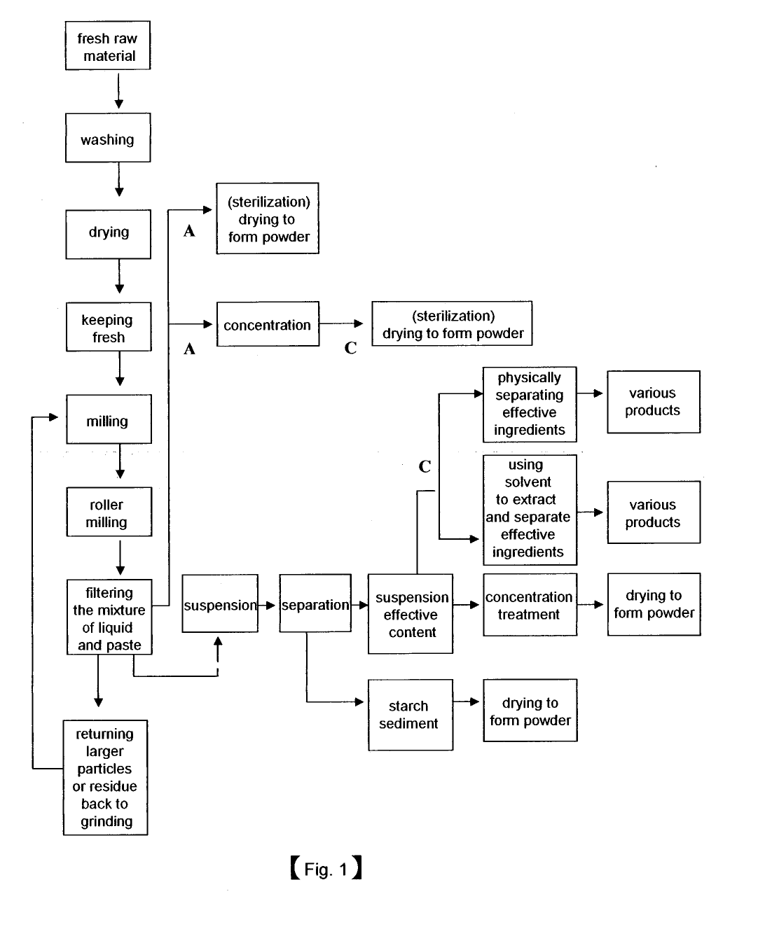

Please refer to FIG. 1, in which fresh raw material, after being harvested, is washed (using water, water spraying, and/or supersonic-style washing) to remove dirts, dehydrated, cut into small pieces (with or without this step), kept fresh (using vacuum forming or low temperature treatment), then subjected to the treatment by millers, rollers, grinders, roller millers, and/or kneaders, to form a mixture of liquid and paste. Then, the mixture of liquid and paste is subjected to one of the following three options of treatment.

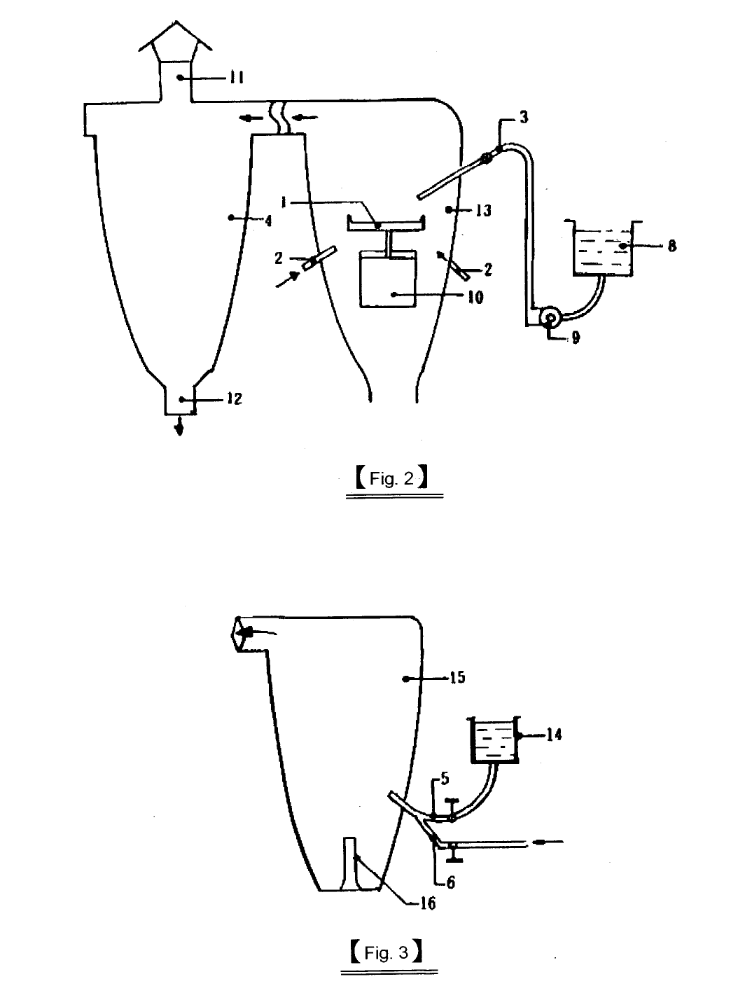

The first option is directly subject the mixture of liquid and paste to drying and powder-forming (as shown in FIGS. 2 or 3), or drying and powder-forming process, sterilization (e.g. ultraviolet light, high temperature treatment, gamma-ray radiation).

The second option is to separate the starch and other material from the mixture of liquid and paste using centrifugal separation or sedimentation separation, and then to subject a suspension fluid on the upper part and the starch and other material to drying treatment to form powders, sterilization treatment can be added to the drying treatment.

The third option is to subject the semi-final products obtained after the separation process (mentioned in the second option) to the treatments such as physically separating effective ingredients, ionic exchange, using solvent to extract and separate effective ingredients, and/or distillation, so as to obtain desirable ingredients.

After the treatment of one of the above-mentioned options, final products can be collected and packaged. The HPLC analysis value of the final products obtained through the second option is higher than that of the final products obtained through the first option. The starch obtained in the second option can be of variable characteristics and utility.

In FIG. 1, the filtering of the mixture of liquid and paste can be fulfilled using e.g. 200 MESH, 300 MESH, or other kind of MESH. It can be a continuous screen or other physical means (e.g. centrifugal means), to remove relatively large pieces of particles or fiber. These relatively large pieces of particles or fiber can be discarded, put back to the machines to be milled or grinded (recycled), or mixed with the raw material and be treated. Furthermore, the steps such as keeping fresh, milling, roller milling can be optional.

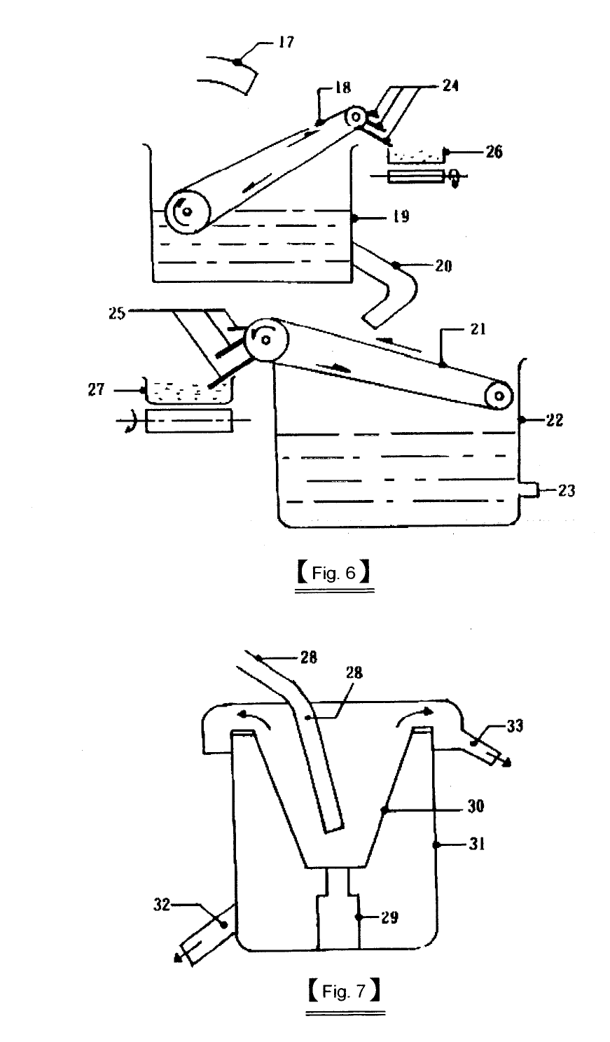

There is no contamination occurred in the process of the present invention because it is done in a closed environment. In FIG. 6, a mixture of liquid and paste flows from a delivery tube 17 toward a continuous (conveyor style) screen 18. The particles which are fine enough flow into a container 19 and then through a tube 20 toward another screen 21. The particles which are fine enough and water then flow into another container 22 and flow out of an exit 23 for the treatment of next step. In this embodiment, there are two steps of screening process. In other embodiments, there may be only one step of screening process, or more than two steps of screen process. The relatively large particles and/or fiber and/or debris are scraped by a scraper 24 or 25 and flow toward a conveyor 26 or 27 and can be treated (milled or grinded) repeatedly.

In FIG. 7, a mixture of liquid and paste flows from a delivery tube 28 toward a rotating funnel-shaped screen 30 which is rotated by a motor 29. The particles which are small enough and water flow downwardly to a container 31 and flow out through an exit 32. The relatively large particles and/or fiber and/or debris fly upwardly and flow out through an exit 33, and can be treated (milled or grinded) repeatedly.

In FIG. 2, the suspended fluid of the mixture of liquid and paste, after being screened, flows from a storage tank 8, through a pump 9, and a tube 3, and toward a high-speed rotating plate 1 (which is rotated by a motor 10 through a shaft). The rotating plate 1 has grooves (having preferably radial or irregular grooves) so as to disintegrate the suspension fluid into fog-style particles. This rotating plate 1 and the motor 10 can be replaced by a device (such as ultra-sonic vibrating device), in another embodiment, to transform the suspension fluid into fog-style particles. Drying air flows from a tube 2 toward an inner face of a housing 13 and toward the fog-style particles of the suspension fluid so as to make the particles dry, and collected by a cyclone 4. The air flows out of a vent 11, and the products (dried fine particles) flow out of an exit 12.

The housing 13 can has a closed upper portion (i.e. without a vent 11, cyclone 4, and exit 12), in another embodiment. In this embodiment, the products (dried fine particles) can flow out from the lower portion of the housing 13.

In FIG. 3, the suspension fluid of the mixture of liquid and paste, after being screened, flows from a storage tank 14, through a tube 5, and is injected toward a chamber 15 by a compressed air flow from a tube 6. Besides, drying air is introduced from a tube 16 into the chamber 15 so that the fine particles can be separated from the water and the air, and collected.

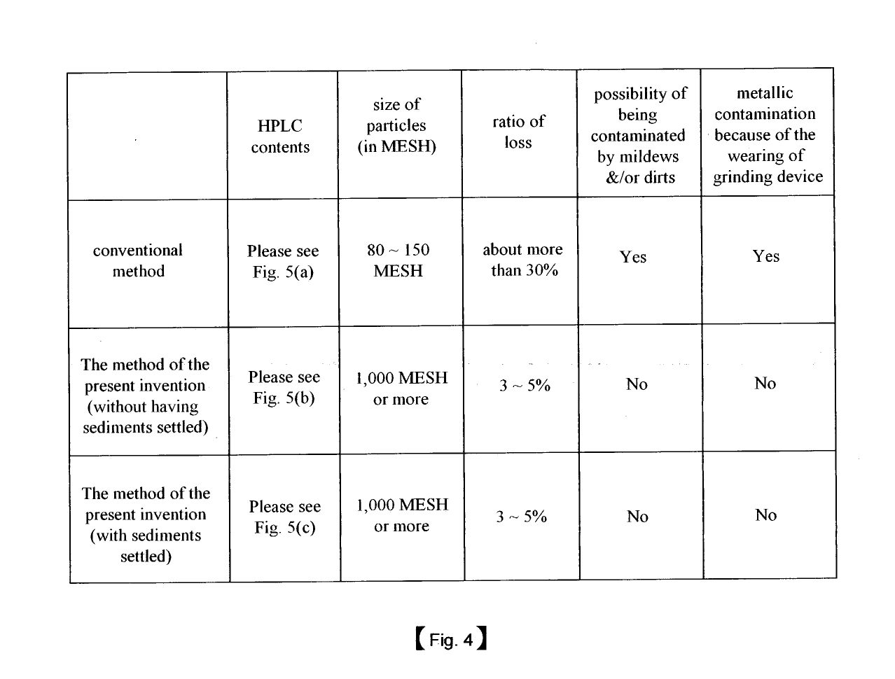

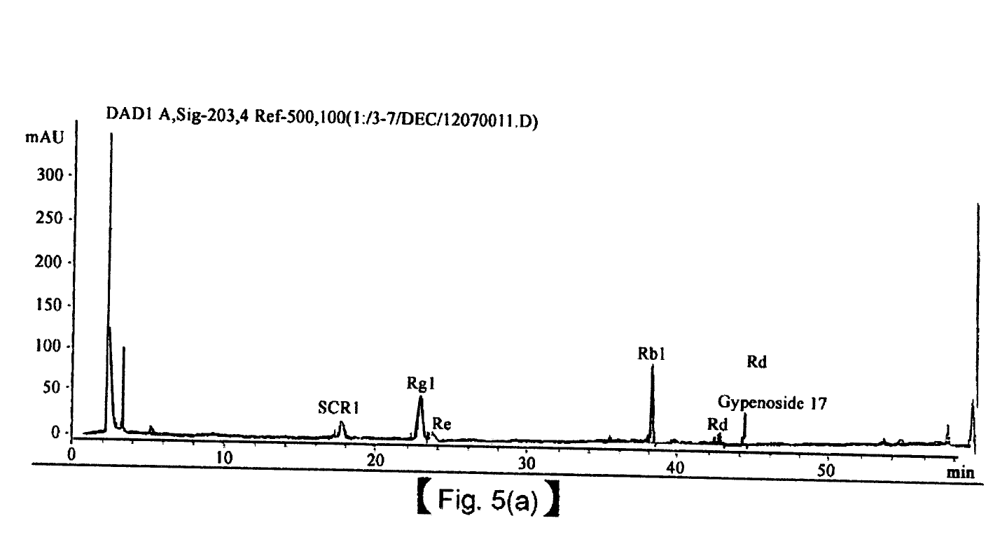

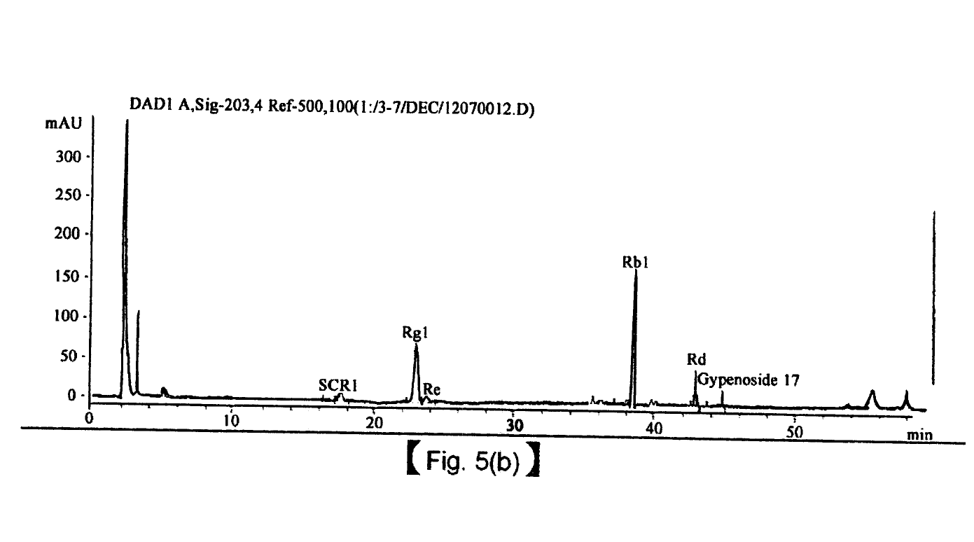

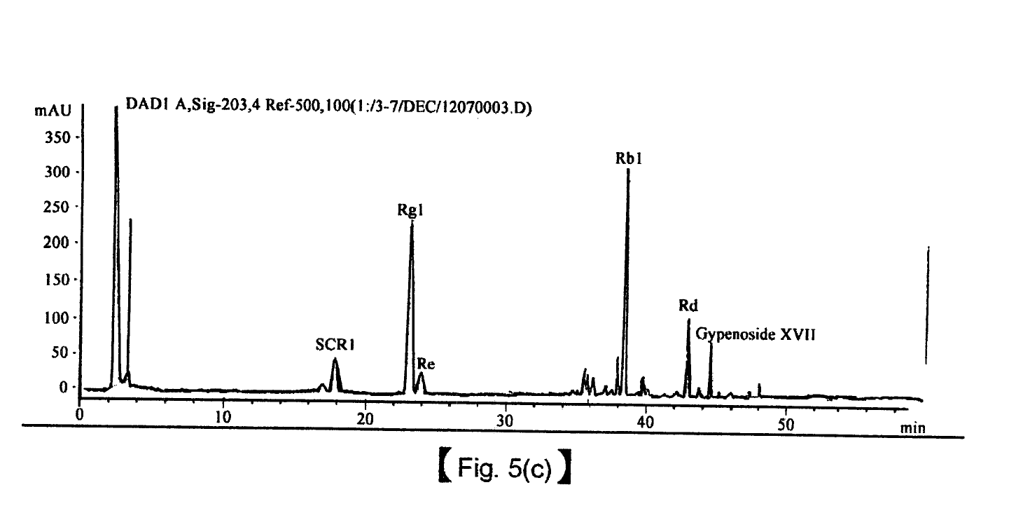

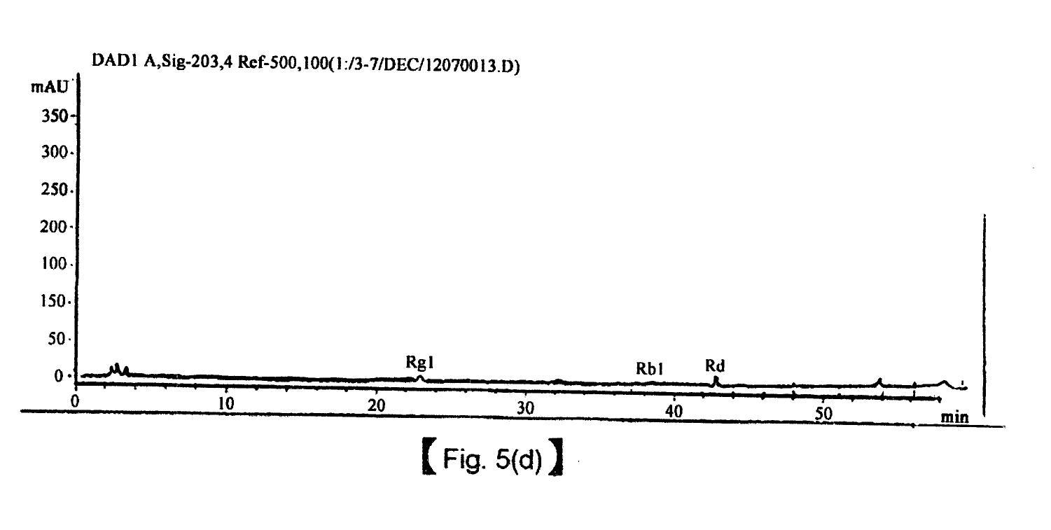

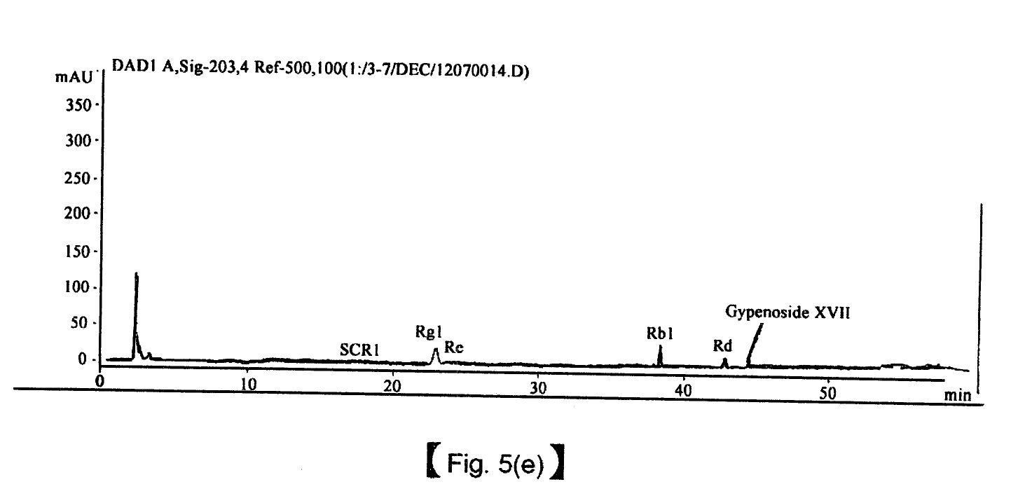

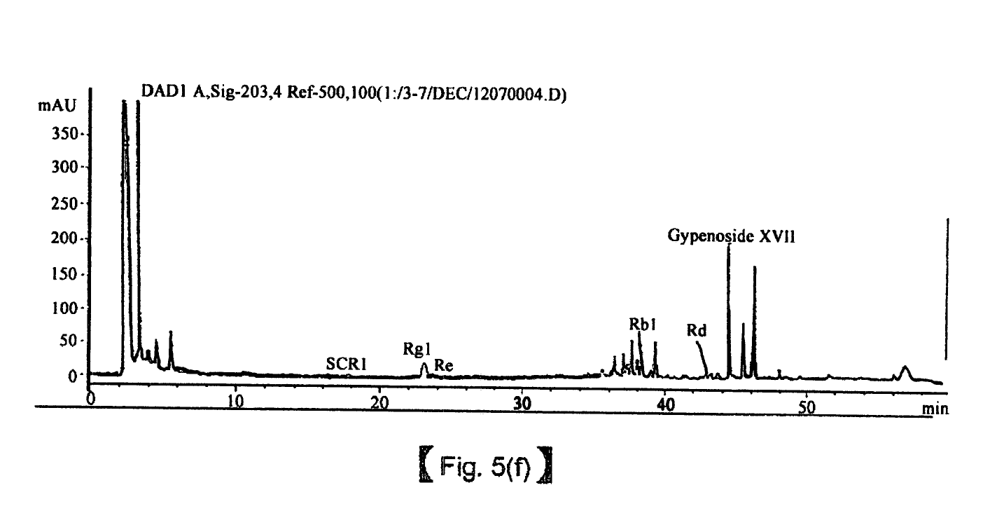

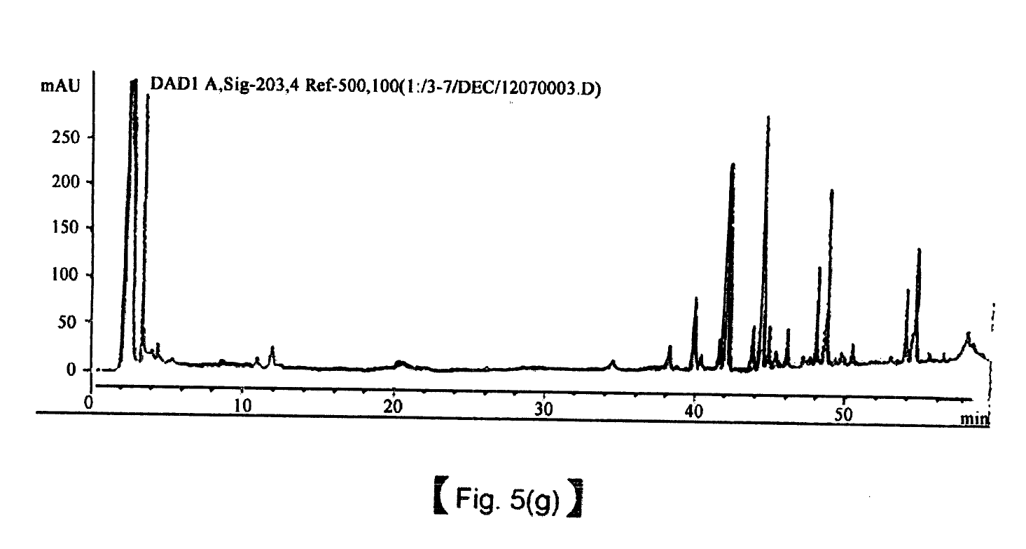

Therefore, the products (particles) produced according to the present invention are very fine (Please refer to FIG. 4, which is 1000 MESH or more, and which is easier to be digested and absorbed by user), are high in effective ingredients (Please see FIG. 5, in which FIG. 5(a) relates to conventional technique, FIG. 5(b) relates to the present invention without the process of sedimentation, FIG. 5(c) relates to the present invention with the process of sedimentation. Furthermore, the method according to the present invention has very low rate of loss, is not susceptible to the human contamination, environmental contamination, or the metallic contamination (because the milling or grinding process is done before the raw material is dried). Also, the method according to the present invention can easily keep the freshness of the material and preserve active cells. The method according to conventional technique does the milling or grinding process after the raw material is dried (which becomes very hard) and thus the machinery is susceptible to frictional wearing and the products (particles or powders) may be polluted by metallic particles.

The method according to the present invention can be used not only to treat (process) the roots of the ginseng or the like, it can be used but also the stems, the leaves of the same, or the roots, the stems, the leaves of other raw material other than the ginseng or the like. The leaves, after being processed with the method of the present invention, can preserve all of the effective ingredients such as chlorophyll and others, and also can preserve the freshness thereof for the use such as being applied to the injuries of human or animals, or other purposes. Conventionally, the leaves are subjected to extraction with alcohols, after being heated, so as to get the effective ingredients. However, a lot of effective ingredients are either damaged or can not be extracted because they are not soluble in alcohols.

FIG. 5(a) to 5(g) show the comparisons of the analysis of the effective ingredients (ginsenosides) obtained through different situations according to the conventional method and the method according to the present invention. FIG. 5(a) shows the ingredients analysis of the products made according to conventional method; FIG. 5(b) shows the ingredients analysis of the products (using roots as raw material and without the process of sedimentation) made according to the present invention; FIG. 5(c) shows the ingredients analysis of the products (using roots as raw material and with the process of sedimentation) made according to the present invention; FIG. 5(d) shows the ingredients analysis of the products of the sediments (using roots as raw material and with the process of sedimentation and separation) made according to the present invention; FIG. 5(e) shows the ingredients analysis of the products of a layer of material located between the sediments and liquid (using roots as raw material and with the process of sedimentation and separation) made according to the present invention; FIG. 5(f) shows the ingredients analysis of the products (using leaves as raw material) made according to the present invention; and FIG. 5(g) shows the ingredients analysis of the products (using leaves as raw material, the leaves are subjected to heating and then extraction using alcohols) according to conventional method.

1. A method for processing biochemical fine powders of ginseng or the like to obtain effective ingredients, mainly including the following steps:
(1) harvesting a raw material, washing the raw material and dehydrating the same;
(2) forming the raw material into a mixture of liquid and paste by using millers, rollers, roller millers and/or kneaders;
(3) filtering the mixture of liquid and paste to form a suspension; and
(4) drying the suspension to form very fine powders.

2. The method according to claim 1, wherein an additional step of cutting the raw material after the first step mentioned in claim I is added; and wherein another additional step of sedimentation and separation after the third step mentioned in claim I is added so that the starch and other ingredients in the mixture of liquid and paste can be sedimented and separated.

3. The method according to claim 1, wherein an additional step of keeping the raw material fresh after the first step mentioned in claim I is added.

4. The method according to claim 1, wherein an additional step of recycling a debris (large particles and fiber) resulted from the filtering after the third step is added so that the debris can go through the second step and the following steps.

5. The method according to claim 1, wherein all or some of the additional steps described herein below are added after the third step: making the starch and other ingredients in the mixture of liquid and paste sedimented; separating the starch and other ingredients using a centrifugal filtering, high density filtering or other filtering method; concentrating a suspension resulted from the separating step (e.g. low temperature low pressure treatment); and using extraction with solvent, distillation, or physical separation to obtain effective ingredients.

6. The method according to claim 1, wherein an additional step of concentrating is added before the fourth step; and an additional step of sterilization is added simultaneously with or after the fourth step.

7. The method according to claim 1, wherein the very fine powders resulted from the fourth step are collected and packaged.

8. The method according to claim 1, wherein ionic exchange processing is used to remove the contamination such as heavy metallic contamination.
